(19) United States
(12) Patent Application Publication
(10) Pub. No.: US 2025/0152879 A1
(43) Pub. Date: May 15, 2025

(54) SYSTEMS AND METHODS FOR VENTILATOR MANAGEMENT

(71) Applicant: KONINKLIJKE PHILIPS N.V., EINDHOVEN (NL)

(72) Inventors: MARIAN DEKKER, EINDHOVEN (NL); PASCAL DE GRAAF, VLEUTEN (NL)

(21) Appl. No.: 18/722,218

(22) PCT Filed: Dec. 19, 2022
(86) PCT No.: PCT/EP2022/086780

§ 371 Date: Jun. 20, 2024

Related U.S. Application Data
(60) Provisional application No. 63/291,488, filed on Dec. 20, 2021.
(60) Provisional application No. 63/291,596, filed on Dec. 20, 2021.
(60) Provisional application No. 63/405,011, filed on Sep. 9, 2022.
(60) Provisional application No. 63/405,015, filed on Sep. 9, 2022.

(51) Int. Cl. A61M 16/00; G16H 40/63; G16H 50/30

(52) U.S. Cl. CPC A61M 16/024; A61M 16/0003; G16H 40/63; G16H 50/30; A61M 2202/0007; A61M 2202/02; A61M 2205/3303; A61M 2205/3327; A61M 2205/3331; A61M 2205/502; A61M 2205/583; A61M 2209/088; A61M 2210/10; A61M 2230/005; A61M 2230/06; A61M 2230/08; A61M 2230/20; A61M 2230/42

(57) ABSTRACT

A system for managing mechanical ventilation provided to a patient by a ventilator. The system includes a plurality of electrodes for being adhered to the patient's chest and a controller in communication with the plurality of electrodes, the controller being programmed to determine from signals received from the electrodes metrics of the patient including: a measure of breathing effort of the patient, and one or both of a heart rate of the patient and/or a respiration rate of the patient. The system further includes a user interface in communication with the controller. The controller is adapted to communicate the metrics to the user interface which is adapted to receive and concurrently display a representation of the measure of breathing effort of the patient on the display; and/or control operation of the ventilator and utilize the measure of breathing effort of the patient in a control algorithm governing such operation.

### BACKGROUND OF THE INVENTION

### 1. Field of the Invention

The present invention pertains to systems and methods for use in managing mechanical ventilation provided to a patient, and further to systems and methods for weaning a patient from mechanical ventilation.

### 2. Description of the Related Art

Proper management of mechanical ventilation provided to a patient is critical for obtaining a positive outcome for the patient. An important part of managing mechanical ventilation is weaning a patient a patient from mechanical ventilation. Weaning from mechanical ventilation is the process used to describe the gradual decrease in ventilatory support provided to a patient. It is estimated that 40% of the duration of mechanical ventilation provided to a patient is dedicated to the process of weaning the patient from the mechanical ventilation. Clinicians face a delicate balance managing ventilatory support provided to a patient and evaluating the readiness of a patient for weaning from the ventilatory support. Failure by a clinician to recognize ventilator withdrawal potential of a patient may result in one or more of increased: time on mechanical ventilation, length of hospital stay, risk of complications (e.g., ventilator associated pneumonia (VAP)), mortality, and general costs. Alternatively, overly aggressive weaning attempts place patients at risk as well. For example, a failed extubation is associated with an approximately 7-fold higher risk for VAP and an approximately 3-fold higher mortality risk.

Several measurements are commonly used in the management of mechanical ventilation and in the process of weaning therefrom. Such measurements include breathing rate, inspiratory effort, inspiratory pressure, expiratory pressure, heart rate, etc. If a patient receiving mechanical ventilation seems ready for weaning, a spontaneous breathing trial (SBT) is performed once a day for 30-120 minutes. An SBT assesses a patient's ability to breathe while receiving minimal or no ventilator support. An important criterion of a SBT is the change in work of breathing or distress of the patient. Studies show that current clinical evaluation using the aforementioned measurements has a low sensitivity for correctly identifying patients who are ready for successful weaning compared to protocolized weaning.

In view of at least the foregoing, there exists a need for improved systems and methods used in managing mechanical ventilation as well as in systems and methods used in weaning a patient from mechanical ventilation.

### SUMMARY OF THE INVENTION

Accordingly, it is an object of the present invention to provide improved systems and methods for managing mechanical ventilation and for weaning a patient therefrom.

In one embodiment, a system for managing mechanical ventilation provided to a patient by a ventilator is provided. The system comprises: a plurality of electrodes, each electrode being structured to be selectively adhered to the chest of the patient; a controller in communication with the plurality of electrodes, the controller being programmed to determine from signals received from the plurality of electrodes a plurality of metrics of the patient, the plurality of metrics comprising: a measure of breathing effort of the patient; and one or both of a heart rate of the patient and/or a respiration rate of the patient; and a user interface in communication with the controller, the user interface having a display, wherein the controller is structured to one or both of: communicate the plurality of metrics to the user interface which is structured to receive and concurrently display a representation of the measure of breathing effort of the patient on the display; and/or control operation of the ventilator and utilize the measure of breathing effort of the patient in a control algorithm governing operation of the ventilator.

The controller may be programmed to determine the measure of breathing effort of the patient from measurements of neural respiratory drive obtained from an EMG signal from at least some of the plurality of electrodes. The EMG signal may be obtained from the at least some of the plurality of electrodes when the at least some of the plurality of electrodes are positioned on the second intercostal space of the patient.

The controller may be structured to receive a number of further measurements from the ventilator, the further measurements comprising one or more of: inspiratory effort, inspiratory pressure and/or expiratory pressure of the patient, wherein the controller is further programmed to determine a calculated spontaneous breathing trial score from the plurality of metrics and the further measurements and communicate the calculated breathing trial score to the user interface, and wherein the user interface is structured to display the calculated spontaneous breathing trial score in place of, or in addition to, the representation of the measure of breathing effort of the patient. The further measurements may comprise: inspiratory effort, inspiratory pressure and expiratory pressure of the patient.

The plurality of electrodes may be coupled together in a sensor patch. The system may further comprise an SpO2 sensor coupled together with the plurality of electrodes in the sensor patch. The controller may be further structured to further concurrently display: an SpO2 value of the patient determined from a signal received from the SpO2 sensor; the heart rate of the patient; and the respiration rate of the patient.

The representation of the measure of breathing effort of the patient displayed on the user interface may comprise a numerical value of the measure of breathing effort.

The representation of the measure of breathing effort of the patient displayed on the user interface may comprise a graphical value of the measure of breathing effort.

The representation of the measure of breathing effort of the patient displayed on the user interface may comprise a directional arrow indicating a recent trend of the value of the measure of breathing effort.

The controller may be structured to receive a number of further measurements from the ventilator, the further measurements comprising one or more of: inspiratory effort, inspiratory pressure and/or expiratory pressure of the patient, wherein the controller is further programmed to determine a calculated spontaneous breathing trial score from the plurality of metrics and the further measurements and utilize the spontaneous breathing trial score as the measure of breathing effort of the patient utilized in the control algorithm governing operation of the ventilator.

In another embodiment, a method of managing mechanical ventilation provided to a patient by a ventilator is provided. The method comprises: receiving signals from a plurality of electrodes positioned on the chest of the patient; determining a plurality of metrics of the patient from the plurality of electrodes positioned on the chest of the patient, the plurality of metrics comprising: a measure of breathing effort of the patient; and one or both of a heart rate of the patient and/or a respiration rate of the patient; and one or both of: communicating the plurality of metrics to a user interface structured to receive and concurrently display a representation of the measure of breathing effort of the patient; and/or controlling operation of the ventilator utilizing the measure of breathing effort of the patient in a control algorithm governing operation of the ventilator.

The method may further comprise: positioning a first electrode of the plurality of electrodes on the second intercostal space of the patient; positioning a second electrode of the plurality of electrodes on the second intercostal space of the patient; and positioning a third electrode of the plurality of electrodes on the sternum of the patient. Positioning each of the first, second, and third electrode is carried out by positioning a single patch arrangement of electrodes on the chest of the patient. The method may further comprise determining SpO2 levels of the patient from an SpO2 sensor positioned on the patient.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 shows a system for managing ventilation of a patient in accordance with an example embodiment of the present invention including a non-invasive monitoring system for determining neural respiratory drive (NRD) of a patient in combination with other metrics typically measured non-invasively, in accordance with example embodiments of the present invention;

FIG. 2 is flow chart of a method for managing ventilation of a patient in accordance with example embodiments of the present invention;

FIG. 3 shows an output display in accordance with an example embodiment of the present invention such as can be provided via a user interface such as shown in the system of FIG. 1; and

FIG. 4 is a decision tree showing the general steps/decisions of an algorithm used in automatically adjusting operational settings of a ventilator based on output from a system such as shown in FIG. 1 and/or a method such as shown in FIG. 2 in accordance with an example embodiment of the present invention.

### DETAILED DESCRIPTION OF EXAMPLE EMBODIMENTS

As used herein, the singular form of “a”, “an”, and “the” include plural references unless the context clearly dictates otherwise.

As used herein, the statement that two or more parts or components are “coupled” shall mean that the parts are joined or operate together either directly or indirectly, i.e., through one or more intermediate parts or components, so long as a link occurs.

As used herein, the term “controller” shall mean a number of programmable analog and/or digital devices (including an associated memory part or portion) that can store, retrieve, execute and process data (e.g., software routines and/or information used by such routines), including, without limitation, a field programmable gate array (FPGA), a complex programmable logic device (CPLD), a programmable system on a chip (PSOC), an application specific integrated circuit (ASIC), a microprocessor, a microcontroller, a programmable logic controller, or any other suitable processing device or apparatus. The memory portion can be any one or more of a variety of types of internal and/or external storage media such as, without limitation, RAM, ROM, EPROM(s), EEPROM(s), FLASH, and the like that provide a storage register, i.e., a non-transitory machine readable medium, for data and program code storage such as in the fashion of an internal storage area of a computer, and can be volatile memory or nonvolatile memory.

As used herein, the term “number” shall mean one or an integer greater than one (i.e., a plurality).

As used herein, the term “sniff” shall mean a deep, sharp inhalation that is perceived by a patient to require maximum inhalation effort.

As used herein with respect to calculation of a neural respiratory drive (NRD) index, the term “relative” shall indicate that the NRD index is calculated using attributes of EMG signals sensed during both regular breathing and sniff activity, and the term “absolute” shall indicate that the NRD index is calculated using attributes of EMG signals sensed only during regular breathing activity.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

The present invention, as described in detail herein in connection with particular example embodiments, provides systems and methods for use in directly (i.e., automatically) and/or indirectly (i.e., via human interaction) managing mechanical ventilation provided to a patient by monitoring/employing values of a measure of breathing effort of the patient in addition to traditional metrics (e.g., heart rate, respiration rate, etc.) of the patient in the decision making processes for such management.

Monitoring respiratory muscle activity in accordance with the disclosed systems and methods enables a neural respiratory drive (NRD) index of the patient to be calculated by a controller. NRD index provides an objective measurement of breathing effort and a more accurate depiction of a patient's respiratory status than other non-invasive parameters that may be used to determine a patient's respiratory status, such as respiration rate alone or respiration rate considered in combination with other non-invasive measurements. Example embodiments of the methods and systems disclosed herein determine NRD index using measurements of EMG signals monitored on the inspiratory muscles of the upper chest of a patient, as EMG measurements taken during inhalation are considered indicative of the balance between respiratory muscle load and respiratory muscle capacity. It should be noted that methods and systems for determining NRD invasively are known, but the methods and systems disclosed herein for calculating NRD index utilize only non-invasive sensors such as EMG electrodes and an accelerometer. The NRD index can be thought of as a non-invasive proxy for an invasive NRD measurement.

For economy of disclosure, the phrase “monitoring NRD index” (and variations thereof such as “monitor NRD index”, etc.) is used herein to encompass the actions of monitoring respiratory muscle activity with EMG electrodes and subsequently calculating the NRD index based on the sensed EMG signals. It should be noted that the NRD index is not a value that is directly output by an EMG electrode. The non-invasive nature of the methods and systems disclosed herein for monitoring NRD index allows a variety of other vital signs/metrics of a patient to be monitored in conjunction with NRD index using a single patch.

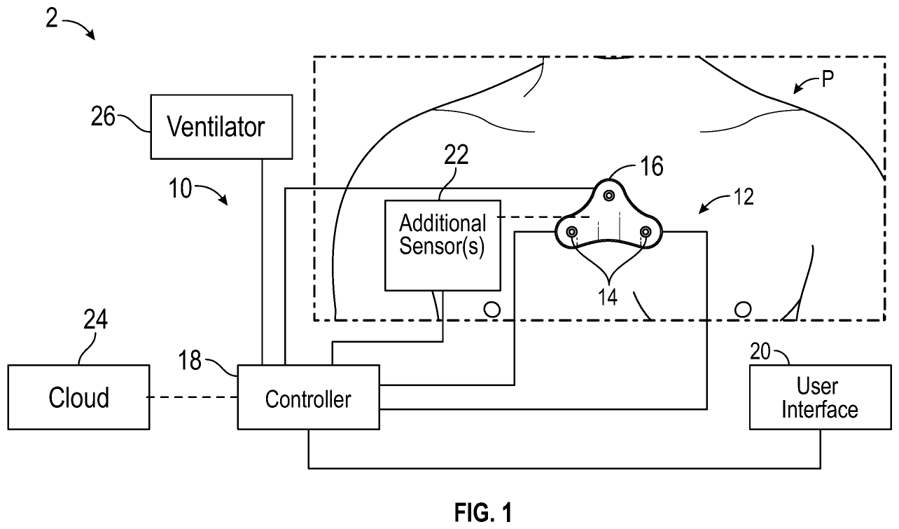

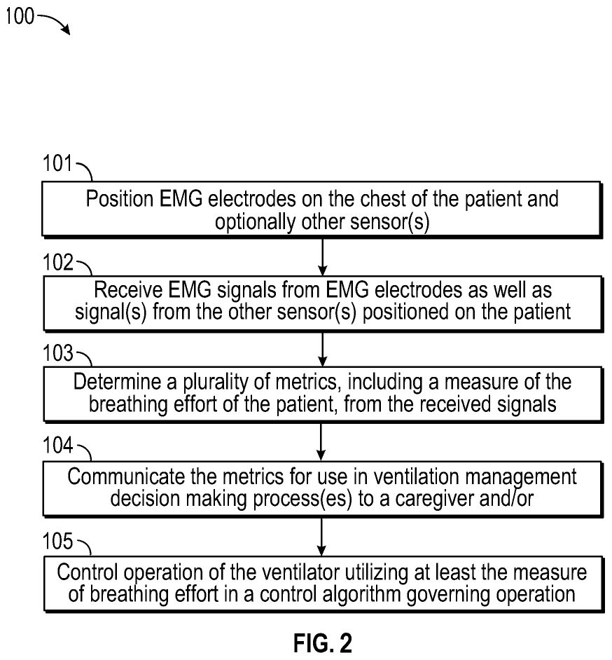

Referring now to FIG. 1, a system 2 for use in managing ventilation of a patient in accordance with an example embodiment of the present invention is shown. System 2 includes a non-invasive patient monitoring system 10 with NRD monitoring capability according to an example embodiment of the present invention. Monitoring system 10 comprises a sensor patch 12, a controller 18, and a user interface 20. Sensor patch 12 comprises a plurality of electrodes, e.g., a number of EMG signal electrodes 14 and a reference electrode 16, and is structured to be affixed to the skin of a patient P via, for example and without limitation, an adhesive layer (not numbered) included on a patient facing side of sensor patch 12. In an example embodiment, sensor patch 12 is structured to be affixed to the upper torso of patient P such that EMG signal electrodes 14 are positioned on the second intercostal space of the patient and reference electrode 16 is placed on the sternum slightly higher (i.e., toward the head of the patient) than EMG signal electrodes 14 in order to optimally monitor respiratory muscle activity of patient P. However, it is to be appreciated that placement of EMG signal electrodes 14 and reference electrode 16 in other locations within the upper chest region suitable for monitoring respiratory muscle activity may be implemented without departing from the scope of the present invention. The EMG signals sensed by EMG signal electrodes 14 are subsequently used to determine NRD index, as discussed later herein with respect to a method 100 depicted in FIG. 2.

Controller 18 is in electrical communication with the electrodes 14 and 16 of sensor patch 12 as well as with user interface 20. Such arrangement of elements enables controller 18 to receive and store the signals measured by EMG signal electrodes 14 and enables a clinician, caregiver, or even patient P to receive the results of any processing performed by controller 18, as well as to input commands to controller 18, via user interface 20. Sensor patch 12 is configured for monitoring a combination of metrics non-invasively, i.e., NRD and at least one other metric, wherein the at least one other metric can comprise, for example and without limitation, heart rate and/or respiration rate (gained from the EMG signal from EMG signal electrodes 14 and 16). Sensor patch 12 may also include a number of additional sensors 22 beyond the EMG electrodes 14, 16. The number of additional sensors 22 can comprise, for example and without limitation, one or more of an accelerometer, a chest SpO2 sensor, a core temperature sensor, and/or other suitable sensors and/or sensor arrangements. It is to be appreciated that while the depiction of additional sensors 22 in FIG. 1 may convey the impression that additional sensors 22 are external to sensor patch 12, it should be noted that such depiction of additional sensors 22 in FIG. 1 is for the purpose of clearly establishing that sensor patch 12 may include one or more additional sensors beyond electrodes 14 and 16 and that such additional sensors 22 may be integrated with sensor patch 12.

User interface 20 may generally be any suitable arrangement for providing information to (e.g., without limitation, a display), and/or receiving information from (e.g., a touchscreen, keyboard, microphone, etc.) a clinician, caregiver, patient, etc. Regarding communication between controller 18 and user interface 20, while controller 18 and user interface 20 are depicted as two separate entities in FIG. 1, it should be noted that in some example embodiments user interface 20 may be integrated into the same physical structure as controller 18, while in other example embodiments controller 18 and user interface 20 exist in separate structures. For example, without limitation, in a hospital setting, user interface 20 can comprise a patient monitor or any other suitable arrangement, while in an embodiment of NRD monitoring system 10 implemented for at-home use, user interface 20 can comprise a smartphone, tablet computer, or any other suitable arrangement. It will be appreciated that controller 18 may be implemented on-board sensor patch 12 or externally to sensor patch 12. Controller 18 can optionally be configured to link the data sensed by sensor patch 12 (and/or additional sensor(s) 22) to a computing cloud 24, such as shown in the example embodiment of FIG. 1.

While FIG. 1 depicts connections between EMG electrodes 14, 16 and controller 18 (and similarly between additional sensors 22 and controller 18), as well as connections between controller 18 and user interface 20, it should be noted that the connections shown are solely intended to depict electrical communication between such respective elements and that such communication can be facilitated via either wired or wireless communication means without departing from the scope of the present invention. In some example embodiments, sensor patch 12 can be a passive patch such that measurements can only be taken when external power is provided to patch 12, and in other example embodiments, sensor patch 12 can be an active patch that includes an onboard power supply and processing means. In one non-limiting example implementation of sensor patch 12 as an active patch, controller 18 is integrated into sensor patch 12 such that the aforementioned onboard power supply and processing means are provided by controller 18. In such example embodiment, controller 18 and user interface 20 are configured to communicate via wireless means such that a care provider or patient can review the results of any processing performed by controller 18.

System 10 may further include a ventilator 26, such as shown in FIG. 1, that is used to provide mechanical ventilation to patient P. In such embodiment, ventilator 26 is in communication (e.g., via suitable wired or wireless means) with controller 18 such that ventilator 26 can communicate information to controller 18 regarding patient P and/or details regarding the ventilation provided to patient P (e.g., inspiratory pressure, expiratory pressure) and/or controller 18 can communicate instructions/commands to ventilator 26 to adjust the ventilation provided by ventilator 26 to patient P. In such example, controller 18 and/or user interface 20 may be integrated with ventilator 26 such that in addition to the functionality previously discussed, controller 18 also controls the operation of one or more components or has the ability to control the operation of the entirety of ventilator 26 while user interface 20 provides for inputting information to, and/or receiving information from, ventilator 26.

Referring now to FIG. 2, a flow chart of a general method 100 for managing ventilation of a patient in accordance with example embodiments of the present invention is shown. Method 100 can be executed, for example, by NRD monitoring system 10 shown in FIG. 1, and thus in such example method 100 is described in conjunction with NRD monitoring system 10. It is to be appreciated, however, that method 100 may be employed with other arrangements without departing from the scope of the present invention.

Method 100 generally begins at step 101 wherein EMG electrodes 14, 16, e.g., without limitation embodied in sensor patch 12, is/are positioned on patient P generally as shown in FIG. 1 (or as otherwise desired) in order to measure respiratory muscle activity of patient P. When an embodiment of NRD monitoring system 10 is being used wherein controller 18 is not implemented on-board sensor patch 12, wires or another suitable communication arrangement is situated between sensor patch 12 and controller 18 during step 101 as well. It is to be appreciated that, in other embodiments, no additional setup by the user is required to establish electrical communication between controller 18 and user interface 20. Such embodiments include, for example and without limitation, those in which a patient monitor includes both controller 18 and user interface 20, or in a home-use context, those in which user interface 20 comprises a smartphone (or other suitable device) and controller 18 comprises a computing cloud in communication with the smartphone. It is to be appreciated that the skin of patient P may need to be prepared at step 101 in order to optimize EMG signal sensing. For example, the skin may need to be cleaned and/or shaved prior to adhering sensors/patch 12 to patient P. In addition to EMG electrodes 14, 16, step 101 may include the placement of one or more additional sensors 22 (e.g., without limitation, an SpO2 sensor) on patient P either included with sensor patch 12 or separate from sensor patch 12. While step 101 is provided in this example embodiment as the first step of method 100, it is to be appreciated that such step may be provided by another in advance without varying from the scope of the present invention and in such example embodiments method 100 would instead begin with step 102 described immediately below.

At step 102, EMG signals produced by patient P (e.g., while support provided to patient P by ventilator 26 is at a minimum) during either or both of regular breathing and/or sniff activity sensed by EMG electrodes 14, 16 (e.g., of sensor patch 12) are received by/in controller 18. In an example embodiment of the present invention, patient P performs one minute of regular breathing followed by one minute of sniffing interspersed with regular breathing, with a sniff being defined as a deep, sharp inhalation that is perceived by patient P to require maximum inhalation effort. In another example embodiment of the present invention, patient P merely breaths regularly while being monitored. In embodiments wherein one or more additional sensors 22 are present on patient P, step 102 may include receiving signals in/by controller 18 from such additional sensor(s) 22 positioned on the patient.

At step 103, a plurality of metrics of the patient, including a measure of breathing effort of the patient, are determined by controller 18 from the signals received in step 102. In an example embodiment of the present invention, the breathing effort of the patient is determined by a number of NRD index values (such as previously described herein) determined by controller 18 based on a number of attributes of the regular breathing and/or sniff EMG signals received at step 102. The NRD index is a quantification of NRD based on various attributes of EMG signals recorded during either or both regular breathing and sniff activity performed by patient P. In addition to such NRD index values, other metrics of the patient (e.g., heart rate, respiration rate) may be determined from the received EMG signals, from the one or more additional sensors 22 (e.g., oxygen saturation from an SpO2 sensor), and/or from ventilator 26 (e.g., inspiratory effort, expiratory pressure). Additionally, one or more advanced metrics (e.g., a calculated SBT score) may be determined from the number of NRD index values and other metrics (e.g., inspiratory effort, expiratory pressure).

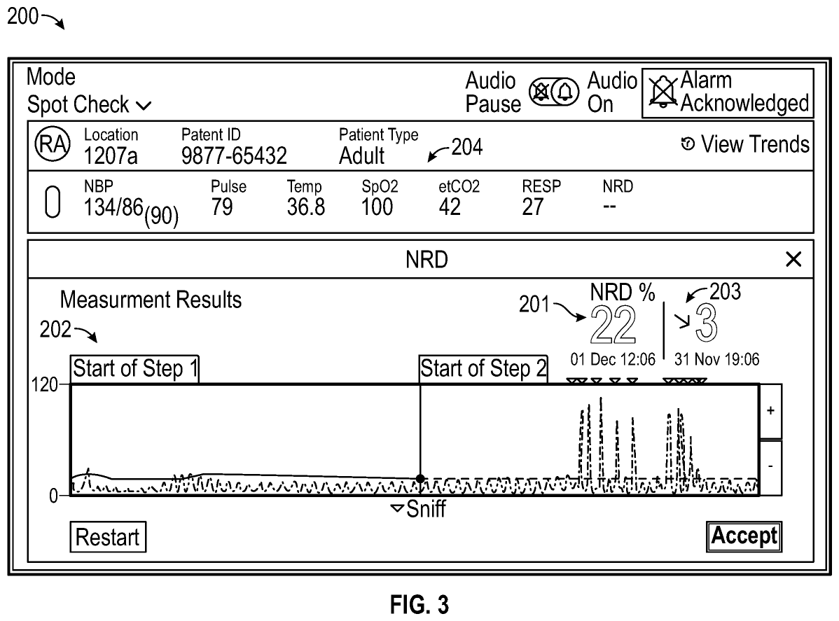

After the metrics are determined at step 103, the metrics may be employed in one or both of two ways. Once such way the metrics may be employed is shown at step 104, wherein the metrics are communicated to a caregiver of the patient for use in ventilation decision making processes. For example, one or more of the metrics may be communicated to interface device 20 where the are displayed on a display thereof for quick reference by a caregiver (doctor, nurse, etc.) who can then employ such information to decide how settings of the ventilator should be adjusted and be able to readily see the effect(s), positive or negative, of changes. An example of such a display 200, in accordance with an example embodiment of the present invention, such as can be provided as a “patient dashboard” on a patient monitor (e.g., located adjacent a hospital bed, in a nursing station, etc.) serving as user interface 20 of system 2 is shown in FIG. 1. Display 200 includes multiple representations of a measure of breathing effort of the patient such as determined in step 103. Such representations of a measure of breathing effort of the patient shown in display 200 include: a numerical value 201 (i.e., NRD % 22), a graph 202 showing fluctuations of values over time, and a progression arrow 203 showing the direction of change in the current value from the last value. Display 200 further includes representations of other patient metrics 204 (e.g., pulse, temp, SpO2, respiration rate, etc.). It is to be appreciated that the values/information/arrangement thereof shown in the example embodiment of FIG. 3 is shown for example purposes only, and that one or both of the information provided therein and/or the arrangement(s) thereof may be varied without varying form the scope of the present invention.

From the example display 200 shown in FIG. 3, it is to be appreciated that by considering a measure of breathing effort of the patient in addition to/concurrently with other metrics of the patient (e.g., heart rate, respiration rate, SpO2, etc.) an improved overall generally instantaneous evaluation of the patient and their reaction(s) to ventilation (and changes thereto) is obtained/provided to the caregiver/clinician from which better informed decisions can be made regarding management of the ventilation. Such evaluation can provide an improved indication of the readiness of the patient for spontaneous breathing and thus weaning from the ventilation.

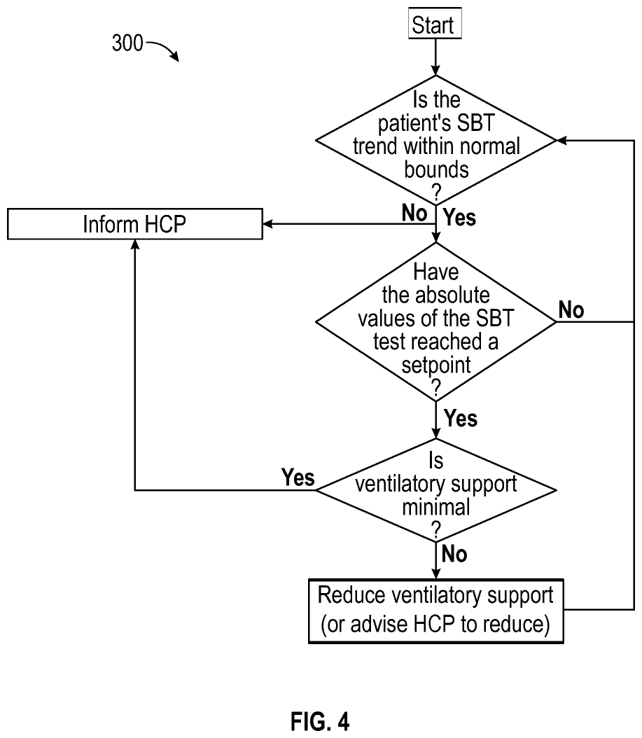

A different or additional way the metrics determined at step 103 may be employed instead of or additionally to that described at step 104 is shown at step 105. In such step the measure of breathing (and optionally other metrics) are utilized in a control algorithm governing operation of ventilator 26. For example, if it is determined from such metric(s) that a patient is ready for weaning, controller 26 could automatically proceed with weaning the patient from ventilation. In reference again to FIG. 1, in such embodiments, instead of (or in addition to) providing indications to a patient and/or caregiver via user interface 20, controller 18 directly controls and/or provides instructions/commands to ventilator 26. Hence, it is to be appreciated that in such arrangement of system 2, operation of ventilator 26 is dependent on the determined measure of breathing effort of the patient. An example of a decision tree 300 showing the general steps/decisions of an algorithm used by controller 18 in automatically adjusting operational settings of a ventilator based on output from a system such as shown in FIG. 1 and/or a method such as shown in FIG. 2 in accordance with an example embodiment of the present invention is shown in FIG. 4.

From the foregoing description and illustrated examples it is thus to be appreciated that by considering a measure of breathing effort of the patient in addition to other metrics of the patient (e.g., heart rate, respiration rate, SpO2, etc.) an improved overall generally instantaneous evaluation of the patient is obtained and then may be employed in making improved decisions regarding managing ventilation provided to the patient.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A system for managing mechanical ventilation provided to a patient by a ventilator, the system comprising:
a plurality of electrodes, each electrode being structured to be selectively adhered to the chest of the patient;
a controller in communication with the plurality of electrodes, the controller being programmed to determine from signals received from the plurality of electrodes a plurality of metrics of the patient, the plurality of metrics comprising:
a measure of breathing effort of the patient; and
one or both of a heart rate of the patient and/or a respiration rate of the patient; and
a user interface in communication with the controller, the user interface having a display,
wherein the controller is structured to one or both of:
communicate the plurality of metrics to the user interface which is structured to receive and concurrently display a representation of the measure of breathing effort of the patient on the display; and/or
control operation of the ventilator and utilize the measure of breathing effort of the patient in a control algorithm governing operation of the ventilator.

2. The system of claim 1, wherein the controller is programmed to determine the measure of breathing effort of the patient from measurements of neural respiratory drive obtained from an EMG signal from at least some of the plurality of electrodes.

3. The system of claim 2, wherein the EMG signal is obtained from the at least some of the plurality of electrodes when the at least some of the plurality of electrodes are positioned on the second intercostal space of the patient.

4. The system of claim 1, wherein the controller is structured to receive a number of further measurements from the ventilator, the further measurements comprising one or more of: inspiratory effort, inspiratory pressure and/or expiratory pressure of the patient,
wherein the controller is further programmed to determine a calculated spontaneous breathing trial score from the plurality of metrics and the further measurements and communicate the calculated breathing trial score to the user interface, and
wherein the user interface is structured to display the calculated spontaneous breathing trial score in place of, or in addition to, the representation of the measure of breathing effort of the patient.

5. The system of claim 4, wherein the further measurements comprise: inspiratory effort, inspiratory pressure and expiratory pressure of the patient.

6. The system of claim 1, wherein the plurality of electrodes are coupled together in a sensor patch.

7. The system of claim 6, further comprising an SpO2 sensor coupled together with the plurality of electrodes in the sensor patch.

8. The system of claim 7, wherein the controller is further structured to further concurrently display:
an SpO2 value of the patient determined from a signal received from the SpO2 sensor;
the heart rate of the patient; and
the respiration rate of the patient.

9. The system of claim 1, wherein the representation of the measure of breathing effort of the patient displayed on the user interface comprises a numerical value of the measure of breathing effort.

10. The system of claim 1, wherein the representation of the measure of breathing effort of the patient displayed on the user interface comprises a graph showing fluctuations of the value of the measure of breathing effort over time.

11. The system of claim 1, wherein the representation of the measure of breathing effort of the patient displayed on the user interface comprises a progression arrow showing the direction of change in the current value from the last value of the measure of breathing effort.

12. The system of claim 1, wherein the controller is structured to receive a number of further measurements from the ventilator, the further measurements comprising one or more of: inspiratory effort, inspiratory pressure and/or expiratory pressure of the patient,
wherein the controller is further programmed to determine a calculated spontaneous breathing trial score from the plurality of metrics and the further measurements and utilize the spontaneous breathing trial score as the measure of breathing effort of the patient utilized in the control algorithm governing operation of the ventilator.

13. A method of managing mechanical ventilation provided to a patient by a ventilator, the method comprising:
receiving signals from a plurality of electrodes positioned on the chest of the patient;
determining a plurality of metrics of the patient from the plurality of electrodes positioned on the chest of the patient, the plurality of metrics comprising:
a measure of breathing effort of the patient; and
one or both of a heart rate of the patient and/or a respiration rate of the patient; and
one or both of:
communicating the plurality of metrics to a user interface structured to receive and concurrently display a representation of the measure of breathing effort of the patient; and/or
controlling operation of the ventilator utilizing the measure of breathing effort of the patient in a control algorithm governing operation of the ventilator.

14. The method of claim 13, further comprising:
positioning a first electrode of the plurality of electrodes on the second intercostal space of the patient;
positioning a second electrode of the plurality of electrodes on the second intercostal space of the patient; and
positioning a third electrode of the plurality of electrodes on the sternum of the patient.

15. The method of claim 14, wherein positioning each of the first, second, and third electrode is carried out by positioning a single patch arrangement of electrodes on the chest of the patient.

16. The method of claim 15, further comprising determining SpO2 levels of the patient from an SpO2 sensor positioned on the patient.
